(19) United States
(12) Patent Application Publication
(10) Pub. No.: US 2025/0143931 A1
(43) Pub. Date: May 8, 2025

(54) Foot Ulcer Healing Cushion Pad Device

(71) Applicant: Diana L. Blanco, Valencia, CA (US)

(72) Inventor: Diana L. Blanco, Valencia, CA (US)

(21) Appl. No.: 18/885,970

(22) Filed: Sep. 16, 2024

Related U.S. Application Data
(60) Provisional application No. 63/595,474, filed on Nov. 2, 2023.

(51) Int. Cl. A61F 13/06

(52) U.S. Cl. CPC A61F 13/063; A61F 13/067

(57) ABSTRACT

A foot ulcer cushion pad device designed for alleviating pressure on ulcers or wounds located on the foot for promoting efficient healing is disclosed here. The cushion pad includes a circular, gel-filled body with a central aperture tailored to encircle and provide relief to an ulcer. The circular body includes detachable first and second horseshoe-shaped portions. Each portion is individually sealed to prevent gel leakage, ensuring durability and effectiveness. The device can be used as a circular device or as an individual horseshoe-shaped device. The device features a substantially planar bottom surface equipped with a skin-friendly, repositionable adhesive layer for secure attachment without residue, and is infused with ulcer healing agents. The device can be used during walking and while wearing any type of the footwear.

### CROSS-REFERENCE TO RELATED APPLICATION

The present application claims priority to, and the benefit of, U.S. Provisional Application No. 63/595,474, which was filed on Nov. 2, 2023, and is incorporated herein by reference in its entirety.

### FIELD OF THE INVENTION

The present invention generally relates to the field of the foot ulcers healing device. More specifically, the present invention relates to a novel cushion pad which is in the shape of a circular ring and has a central aperture. The pad utilizes a gel-filled structure that can alleviate pressure and friction on the ulcer to facilitate faster healing. The device can have an adhesive layer for adherence to the skin of the foot of an individual. The cushion pad is placed around an ulcer to provide cushion, reducing pressure and friction on the skin to help the ulcer heal. Accordingly, the present disclosure makes specific reference thereto. Nonetheless, it is to be appreciated that aspects of the present invention are also equally applicable to other like applications, devices, and methods of manufacture.

### BACKGROUND

By way of background, individuals with diabetes or with poor blood circulation may have trouble with ulcers on the bottoms of their feet. Diabetes can cause nerve damage which is commonly called neuropathy. Neuropathy can lead to a loss of feeling in the feet, making it difficult to notice cuts, blisters, or other injuries. The unnoticed injuries can worsen into ulcers. Poor circulation reduces the amount of oxygen and nutrients reaching the feet. In severe cases, poor circulation can lead to gangrene and tissue death that may require amputation.

Further, constant pressure and friction on the feet such as from poorly fitting shoes or walking barefoot, can aggravate existing wounds or lead to the formation of ulcers. Ulcers are often not properly covered or relieved of pressure and thus become larger and deeper. Also, ulcers are continually subjected to pressure and stress due to their location on the bottom of the feet, which can interrupt the healing process. Additionally, for individuals with diabetes or poor circulation, the reduced blood flow further slows down the healing of the wounds. When untreated, the skin and tissue around the ulcer can become infected, leading to conditions like cellulitis, or the infection can penetrate deeper, affecting the bones (osteomyelitis) and leading to serious health complications.

Individuals often resort to ointments and other medical treatments for treating ulcers. However, such methods are expensive, time consuming and may require a medical device. Individuals desire an improved way of continuously taking pressure off an ulcer and reducing friction on the ulcer to facilitate faster healing of the ulcer.

Therefore, there exists a long felt need in the art for a device that can be applied to a foot area to alleviate pressure from an ulcer to heal the ulcer. There is also a long felt need in the art for a cushion pad device that can be applied around an ulcer on the foot area of a user. Additionally, there is a long felt need in the art for a novel cushion device that is configured to be placed around a wound on the bottom of a foot of a user to reduce friction on the wound. Moreover, there is a long felt need in the art for a novel circular cushion pad device that has a central aperture for accommodating an ulcer and a peripheral gel-filled body for reducing pressure on the ulcer. Further, there is a long felt need in the art for a specially designed gel-filled cushion device that is placed around an ulcer to help the ulcer heal quickly. Furthermore, there is a long felt need in the art for a novel cushion device that eliminates use of expensive ointments and foot area ulcer treatments for healing foot area ulcers quickly. Finally, there is a long felt need in the art for a cushion pad that can be configured to place around a foot ulcer to increase blood circulation and to prevent friction to heal ulcers quickly and easily.

The subject matter disclosed and claimed herein, in one embodiment thereof, comprises a foot ulcer cushion device for alleviating pressure on a foot ulcer or wound. The cushion device includes a circular gel-filled body forming a central aperture, the central aperture accommodates an ulcer or wound and the circular gel-filled body circumscribes the ulcer or wound. The circular body includes a two symmetrical horseshoe-shaped portions, each horseshoe-shaped portion has a first end and an opposing second end, the first horseshoe-shaped portion and the second horseshoe-shaped portion are detachably attached at their corresponding first ends and an opposing ends to form the circular gel-filled body. An adhesive layer is disposed on the bottom surface of the circular body and includes a skin-friendly and repositionable adhesive infused with a mild, skin-regenerating agent to promote healing of the accommodated ulcer.

In this manner, the foot ulcer cushion device of the present invention accomplishes all of the forgoing objectives and provides users with an ulcer cushion that has a gel-filled structure and includes a central aperture for accommodating an ulcer. The cushion pad is circular in shape and has two horseshoe-shaped detachably attached (i.e., selectively attachable) pieces. The cushion device contacts the skin around the ulcer to promote blood circulation and alleviate pressure on the ulcer to heal the ulcer. The horseshoe-shaped pieces can be detached and individually used for providing pressure to heal ulcers. The device reduces use of ointments and other treatments for healing foot ulcers.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a foot ulcer cushion device. The cushion device is designed to place around a foot ulcer to alleviate pressure and friction on the ulcer to facilitate faster healing of the ulcer. The cushion device further comprises a circular body formed as a ring with a central aperture disposed therein, the circular body is placed around an ulcer with the central aperture accommodating the ulcer, the circular body includes a gel-filled construction to provide cushioning, wherein the circular body is divisible into a first horseshoe-shaped portion and a second horseshoe-shaped portion, each portion detachably attached at their respective ends to enable for the device to adapt to various sizes and locations of ulcers or wounds.

In another aspect, the circular body has a selectable thickness up to 0.5 inches to prevent discomfort during use, and wherein the cushion pad device is adapted to be used as a circular device or in part as one or more horseshoe-shaped devices depending on the specific needs of the ulcer or wound treatment, each horseshoe-shaped portion being individually sealed to maintain the gel material therein.

In yet another aspect, the gel material contained within the circular body includes silver nanoparticles to offer antimicrobial resistance, enhancing the healing process of the ulcer or wound by preventing infection.

In yet another embodiment, a foot ulcer cushion device for alleviating pressure on an ulcer or wound located on a user's foot is disclosed. The cushion device includes a circular gel-filled body forming a central aperture, the central aperture accommodates an ulcer or wound and the circular gel-filled body circumscribes the ulcer or wound. The circular body includes a first horseshoe-shaped portion and a second horseshoe-shaped portion, each horseshoe-shaped portion extends from a respective first end to a respective opposite end, wherein the first horseshoe-shaped portion and the second horseshoe-shaped portion are detachably attached at their corresponding first ends and opposing ends to form the circular gel-filled body. Each horseshoe-shaped portion is individually sealed to prevent leakage of the gel contained therein, enabling their individual and independent use for healing ulcers. An adhesive layer is disposed on the bottom surface of the circular body and includes a skin-friendly and repositionable adhesive infused with a mild, skin-regenerating agent to promote healing of the accommodated ulcer.

In a further embodiment, the bottom surface of the circular body is substantially planar to ensure secure and stable placement on the foot, and a top surface of the circular body may be convex or substantially flat based on user preference.

In another aspect, a method for using a foot ulcer cushion device to alleviate pressure and promote healing of ulcers or wounds on a user's foot is described. The method includes the steps of providing a foot ulcer cushion device comprising a circular gel-filled body forming a central aperture, the circular body has a first and a second horseshoe-shaped portion that are detachably connected to form the circular body, wherein each horseshoe-shaped portion is individually sealed and contains gel. The method further includes selecting the configuration of the cushion device based on the location and size of the ulcer or wound, wherein the device can be used in the circular form for encircling the ulcer or wound or as separate horseshoe-shaped portions for targeted pressure relief, applying the circular gel-filled body or separated horseshoe-shaped portions around the ulcer or wound on the foot in order to reduce direct pressure on the ulcer, and securing the cushion device to the foot using an adhesive layer disposed on the bottom surface of the cushion device.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the foot ulcer cushion device of the present invention in accordance with one embodiment of the present invention;

FIG. 2 illustrates a perspective view showing the two horseshoe shaped cushion pads in accordance with one embodiment of the present invention;

FIG. 3 illustrates a bottom perspective view of the foot of a user with application of cushion pads of the present invention for healing ulcers and wounds in accordance with one embodiment of the present disclosure;

FIG. 4 illustrates a perspective view of another embodiment of the foot ulcer cushion device of the present invention in accordance with the disclosed structure; and

FIG. 5 illustrates a side perspective of a user using the foot ulcer cushion pad of FIG. 4 in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a device that can be applied to a foot area to alleviate pressure from an ulcer to heal the ulcer. There is also a long felt need in the art for a cushion pad device that can be applied around an ulcer on the foot area of a user. Additionally, there is a long felt need in the art for a novel cushion device that is configured to place around a wound on bottom of the foot of a user to reduce friction on the wound. Moreover, there is a long felt need in the art for a novel circular cushion pad device that has a central aperture for accommodating an ulcer and a peripheral gel-filled body for reducing pressure on the ulcer. Further, there is a long felt need in the art for a specially designed gel-filled cushion device that is placed around an ulcer to help the ulcer heal quickly. Furthermore, there is a long felt need in the art for a novel cushion device that eliminates use of expensive ointments and foot area ulcer treatments for healing foot area ulcers quickly. Finally, there is a long felt need in the art for a cushion pad that can be configured to place around a foot ulcer to increase blood circulation and to prevent friction to heal ulcers quickly and easily.

The present invention, in one exemplary embodiment, is a foot ulcer cushion device. The cushion device is designed to place around a foot ulcer to alleviate pressure and friction on the ulcer to facilitate faster healing of the ulcer. The cushion device further comprises a circular body formed as a ring with a central aperture disposed therein, the circular body is placed around an ulcer with the central aperture accommodating the ulcer, the circular body includes a gel-filled construction to provide cushioning, wherein the circular body is divisible into a first horseshoe-shaped portion and a second horseshoe-shaped portion, each portion detachably attached at their respective ends to enable for the device to adapt to various sizes and locations of ulcers or wounds.

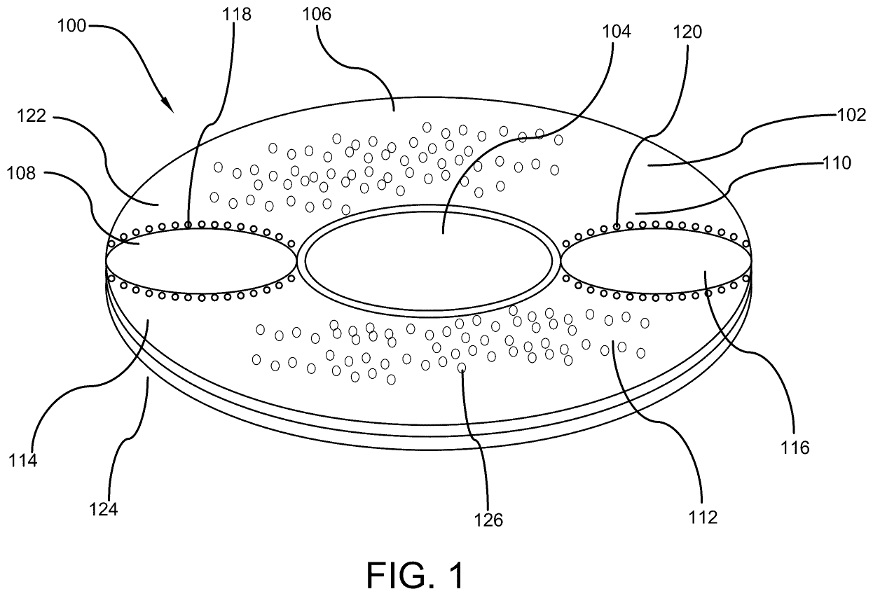

Referring initially to the drawings, FIG. 1 illustrates a perspective view of the foot ulcer cushion device of the present invention in accordance with one embodiment of the present invention. The foot ulcer cushion device 100 of the present invention is designed to be placed around an ulcer or wound on the bottom of the foot of a user. The cushion device 100 reduces pressure directly on the ulcer, which is essential for preventing the wound from worsening and aiding in the healing process of the ulcer. Preferably, the cushion device 100 is a circular device having a gel-filled circular body 102 with a central aperture 104. The circular body 102 is in the form of a ring forming the aperture 104.

The central aperture 104 is adapted to accommodate an ulcer or wound and the circular body 102 circumscribes the accommodated ulcer to alleviate the pressure therefrom. The circular body 102 has a first horseshoe shaped portion 106 extending from a first end 108 to an opposing second end 110. The circular body 102 also has a second horseshoe shaped portion 112 extending from a corresponding first end 114 to an opposing second end 116. The first end 108 of the first horseshoe shaped portion 106 is detachably attached (i.e., selectively attached) to the corresponding first end 114 of the second horseshoe shaped portion 112. The opposite end 110 of the first horseshoe shaped portion 106 is detachably attached to the corresponding second end 116 of the second horseshoe shaped portion 112. The first ends 108, 114 form a continuous first depression line or connection 118 that extends across the top surface 122 and the bottom surface 124 of the circular body 102 and the second ends 110, 116 for a second depression line or connection 120 across the surfaces 122, 124, wherein the first horseshoe shaped portion 106 and the second horseshoe shaped portion 112 are detached along the depression lines 118, 120 when pulled away from each other by a user.

The circular body 102 is sealed thereby eliminating any leakage of gel filled therein. The circular body 102 can have a thickness of up to 0.5 inches to prevent any discomfort during use of the device 100 while alleviating pressure from the accommodated ulcer. The device 100 can be used as a circular device or as a horseshoe shape device according to the location and size of an ulcer. The horseshoe shaped portions 106, 112 are individually sealed and can be used individually and independently for healing ulcers.

The bottom surface 124 of the circular body 102 is substantially planar for a secure and stable placement on the foot of a user and the top surface 122 can be convex or substantially flat depending on the preference of a user. In the present embodiment, the bottom surface 124 creates a vacuum suction to adhere the cushion pad 100 to the foot of a user when the cushion pad 100 is pressed against the foot around an ulcer. The gel material 126 included in the horseshoe shaped portions 106, 112 can be similar or different and the device 100 can be kept in a freezing device for freezing the gel 126 before application on the foot around an ulcer or a wound. The circular body 102 can be transparent or translucent and the gel circular body 102 prevents contact of the ulcer to a surface such as when a user using the device 100 walks on the ground surface.

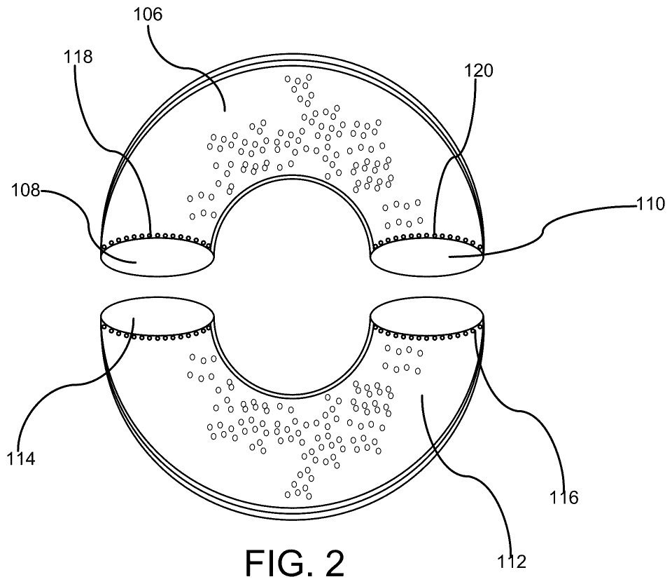

FIG. 2 illustrates a perspective view showing the two horseshoe shaped cushion pads in accordance with one embodiment of the present invention. As illustrated, the cushion device 100 is divided into two horseshoe shaped cushion pads 106, 112 when detached from the cushion device 100. The horseshoe shaped cushion pads 106, 112 can be of similar size or different sizes depending on preferences of user and manufacturing requirements.

In some embodiments, the horseshoe shaped cushion pads 106, 112 can be attached to form the circular cushion pad 100 and alternatively, the horseshoe shaped cushion pads 106, 112 may not attach once detached.

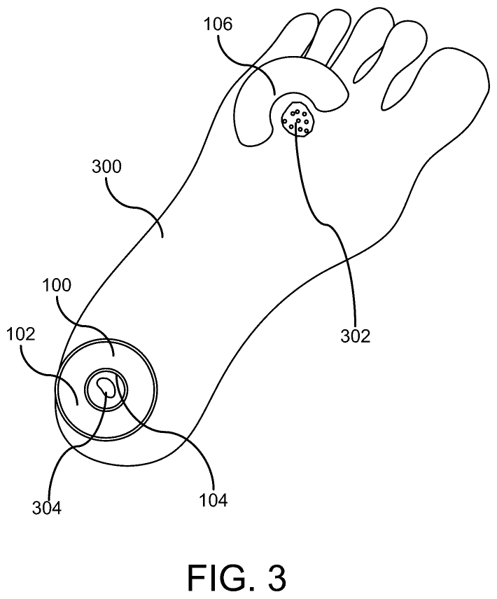

FIG. 3 illustrates a bottom perspective view of the foot of a user with application of cushion pads of the present invention for healing ulcers and wounds in accordance with one embodiment of the present disclosure. As illustrated, a horseshoe shaped pad 106 is positioned around a first ulcer 302 on foot 300 for providing pressure relief and the foot ulcer circular cushion pad 100 is positioned around a second ulcer/wound 304 for providing pressure relief and improving blood circulation. The cushion pads 100, 106 can be used when walking barefoot and during wearing shoes or any footwear as well. The circular cushion body 102 contacts the skin to adjust pressure on the ulcer for promoting healing of the ulcer. Further, the contraction of skin by the circular body 102 helps in improving the circulation in the ulcer to promote healing.

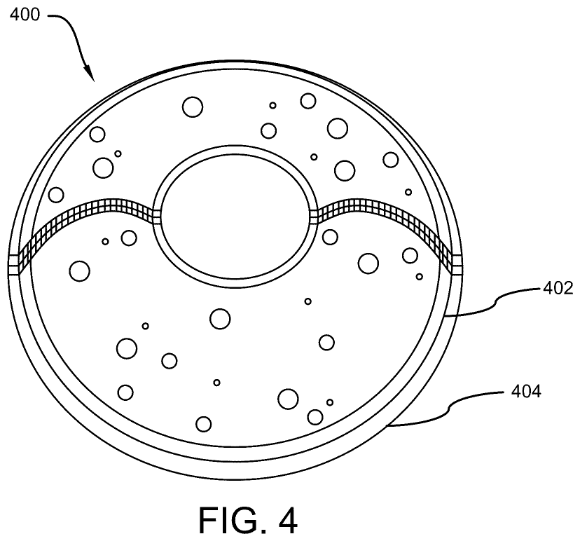

FIG. 4 illustrates a perspective view of another embodiment of the foot ulcer cushion device of the present invention in accordance with the disclosed structure. In the present embodiment, the bottom surface 402 includes a pressure adhesive layer 404 disposed thereon for adhering the cushion device 400 to the skin of the foot of an individual. Preferably, the adhesive layer 404 is in the form of a silicon gel or liquid silicon. Alternatively, the adhesive layer 404 can be in the form of a transparent acrylic tape. All other specifications of the cushion pad 400 remains same as of the cushion pad 100 of FIG. 1.

The adhesive layer 404 on the bottom surface 402 of the cushion pad 400 can employ a skin-friendly and repositionable adhesive that does not lose the grip or leave residue on the skin. The adhesive layer 404 can be infused with a mild, skin-regenerating agent to promote healing of the ulcer accommodated by the cushion device 400.

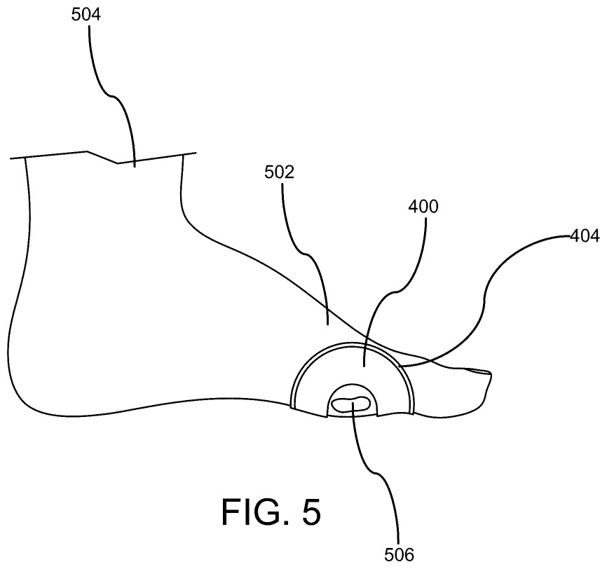

FIG. 5 illustrates a side perspective of a user using the foot ulcer cushion pad of FIG. 4 in accordance with the disclosed structure. As illustrated, the cushion device 400 is adhered to the side 502 of the foot 504 of a user using the adhesive layer 404 for providing a stable position of the cushion device 400 around the ulcer 506. The cushion pad 400 can be easily removed by peeling off the cushion pad 400 from the foot of the user. The pad 100, 400 in different embodiments prevent direct contact of an accommodated ulcer with a surface, thereby reducing friction on the ulcer.

In some embodiments of the present invention, the central aperture 104 of the cushion device 100, 400 can be adjusted by removing the gel from the circular body thereof to accommodate ulcers/wounds of different sizes. The circular body 102 preferably includes silver nanoparticles for improving antimicrobial resistance of the cushion pad device to heal ulcers quickly.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “foot ulcer cushion pad device”, “foot ulcer cushion pad”, “cushion device”, “cushion pad”, and “device” are interchangeable and refer to the foot ulcer healing cushion pad device 100, 400 of the present invention.

Notwithstanding the forgoing, the foot ulcer healing cushion pad device 100, 400 of the present invention can be of any suitable size and configuration as is known in the art without affecting the overall concept of the invention, provided that it accomplishes the above stated objectives. One of ordinary skill in the art will appreciate that the foot ulcer healing cushion pad device 100, 400 as shown in the FIGS. are for illustrative purposes only, and that many other sizes and shapes of the foot ulcer healing cushion pad device 100, 400 are well within the scope of the present disclosure. Although the dimensions of the foot ulcer healing cushion pad device 100, 400 are important design parameters for user convenience, the foot ulcer healing cushion pad device 100, 400 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A foot ulcer cushion device for placing around an ulcer comprising:
a gel-filled circular body circumscribing a circular aperture;
a first horseshoe shaped portion; and
a second horseshoe shaped portion;
wherein said first horseshoe shaped portion extending from a first end to an opposing second end;
wherein said second horseshoe shaped portion extending from a first end to an opposing second end;
wherein said first end of said first horseshoe shaped portion selectively attached to said first end of said second horseshoe shaped portion to form a continuous first depression connection extending across a first top surface and a first bottom surface of said gel-filled circular body;
wherein said second end of said first horseshoe shaped portion selectively attached to said second end of said second horseshoe shaped portion to form a continuous second depression connection across a second top surface and a second bottom surface of said gel-filled circular body;
wherein said first horseshoe shaped portion is a first sealed gel-filled body;
wherein said second horseshoe shaped portion is a second sealed gel-filled body; and
further wherein said first bottom surface and said second bottom surface are planar.

2. The foot ulcer cushion device for placing around an ulcer of claim 1, wherein said first top surface and said second top surface are convex.

3. The foot ulcer cushion device for placing around an ulcer of claim 1, wherein said first top surface and said second top surface are flat.

4. The foot ulcer cushion device for placing around an ulcer of claim 2, wherein said gel-filled circular body is frozen before application to the ulcer of a foot of a user.

5. The foot ulcer cushion device for placing around an ulcer of claim 4, wherein said gel-filled circular body having a material selected from the group consisting of a transparent material and a translucent material.

6. The foot ulcer cushion device for placing around an ulcer of claim 5, wherein said first bottom surface and said second bottom surface having a pressure adhesive layer disposed thereon for adhering said gel-filled circular body to skin of the foot of the user, and further wherein said gel-filled circular body circumscribes the ulcer when adhered to the foot of the user.

7. The foot ulcer cushion device for placing around an ulcer of claim 6, wherein said first horseshoe shaped portion and said second horseshoe shaped portion detachable along said continuous first depression connection and said continuous second depression connection.

8. The foot ulcer cushion device for placing around an ulcer of claim 7, wherein said gel-filled circular body having a thickness up to 0.5 inches.

9. The foot ulcer cushion device for placing around an ulcer of claim 8, wherein said gel-filled circular body having silver nanoparticles for improving antimicrobial resistance of said gel-filled circular body to heal ulcers.

10. A foot ulcer cushion device for placing around an ulcer comprising:
a gel-filled circular body circumscribing a circular aperture;
a first horseshoe shaped portion of a first size; and
a second horseshoe shaped portion of a second size;
wherein said first horseshoe shaped portion extending from a first end to an opposing second end;
wherein said second horseshoe shaped portion extending from a first end to an opposing second end;
wherein said first end of said first horseshoe shaped portion selectively attached to said first end of said second horseshoe shaped portion to form a continuous first depression connection extending across a first top surface and a first bottom surface of said gel-filled circular body;
wherein said second end of said first horseshoe shaped portion selectively attached to said second end of said second horseshoe shaped portion to form a continuous second depression connection across a second top surface and a second bottom surface of said gel-filled circular body;
wherein said first horseshoe shaped portion is a first sealed gel-filled body;
wherein said second horseshoe shaped portion is a second sealed gel-filled body; and
further wherein said first size is different from said second size.

11. The foot ulcer cushion device for placing around an ulcer of claim 10, wherein said first bottom surface and said second bottom surface are planar.

12. The foot ulcer cushion device for placing around an ulcer of claim 10, wherein said first top surface and said second top surface are convex.

13. The foot ulcer cushion device for placing around an ulcer of claim 10, wherein said first bottom surface and said second bottom surface having a pressure adhesive layer disposed thereon for adhering said gel-filled circular body to skin of the foot of the user, and further wherein said gel-filled circular body circumscribes the ulcer when adhered to the foot of the user.

14. The foot ulcer cushion device for placing around an ulcer of claim 10, wherein said gel-filled circular body is frozen before application to the ulcer of a foot of a user.

15. The foot ulcer cushion device for placing around an ulcer of claim 10, wherein said gel-filled circular body having silver nanoparticles for improving antimicrobial resistance of said gel-filled circular body to heal ulcers.

16. The foot ulcer cushion device for placing around an ulcer of claim 10, wherein said first horseshoe shaped portion and said second horseshoe shaped portion detachable along said continuous first depression connection and said continuous second depression connection.

17. A method of treating a foot ulcer, the method comprising the steps of:
providing a gel-filled circular body circumscribing a circular aperture, a first horseshoe shaped portion of a first size, a second horseshoe shaped portion of a second size, wherein said first horseshoe shaped portion extending from a first end to an opposing second end, wherein said second horseshoe shaped portion extending from a first end to an opposing second end, wherein said first horseshoe shaped portion is a first sealed gel-filled body, further wherein said second horseshoe shaped portion is a second sealed gel-filled body;
freezing said gel-filled circular body before application to an ulcer of a foot of a user;
selectively attaching said first end of said first horseshoe shaped portion to said first end of said second horseshoe shaped portion to form a continuous first depression connection extending across a first top surface and a first bottom surface of said gel-filled circular body; and
selectively attaching said second end of said first horseshoe shaped portion to said second end of said second horseshoe shaped portion to form a continuous second depression connection across a second top surface and a second bottom surface of said gel-filled circular body.

18. The method of treating the foot ulcer of claim 17, wherein said first bottom surface and said second bottom surface are planar, and further wherein said first top surface and said second top surface are convex.

19. The method of treating the foot ulcer of claim 18, wherein said first bottom surface and said second bottom surface having a pressure adhesive layer disposed thereon for adhering said gel-filled circular body to skin of the foot of the user, and further wherein said gel-filled circular body circumscribes the ulcer when adhered to the foot of the user.

20. The method of treating the foot ulcer of claim 19, wherein said gel-filled circular body having silver nanoparticles for improving antimicrobial resistance of said gel-filled circular body to heal ulcers.
